(19) United States
(12) Patent Application Publication
(10) Pub. No.: US 2013/0304136 A1
(43) Pub. Date: Nov. 14, 2013

(54) FRANGIBLE FIXING SCREW

(71) Applicants: Jeannine Gourlaouen-Preissler, Montreuil (FR); Bertrand Busson, Paris (FR)

(72) Inventors: Jeannine Gourlaouen-Preissler, Montreuil (FR); Bertrand Busson, Paris (FR)

(73) Assignee: ZDA Zirconia Developpement & Applications, Laval Cedex (FR)

(21) Appl. No.: 13/980,042

(22) PCT Filed: Dec. 30, 2011
(86) PCT No.: PCT/EP2011/074331

§ 371 Date: Jul. 16, 2013

(30) Foreign Application Priority Data
Jan. 18, 2011 (FR) 11/00145

(51) Int. Cl. A61B 17/86

(52) U.S. Cl. CPC A61B 17/8605
USPC 606/305; 470/9

(57) ABSTRACT

The invention relates to a screw, particularly for attaching a superstructure to an intra-osseous implant. The extends along a longitudinal axis and comprises a head and a thread. The head comprises a bearing surface configured to cooperate with a bearing surface of the superstructure to hold the superstructure in position. The thread is configured to engage with a tapping to tighten the bearing surfaces of the superstructure and the screw. Between the bearing surface and the thread, the screw comprises a safety portion. The safety portion comprises a driving shape and a frangible section at the connection between the safety portion and the head of the screw, so that the driving shape remains attached to the thread if the frangible section breaks.

### RELATED APPLICATIONS

This application is a §371 application from PCT/EP2011/074331 filed Dec. 30, 2011, which claims priority from French patent application Ser. No. 11/00145 filed Jan. 18, 2011, each of which is herein incorporated by reference in its entirety.

### TECHNICAL FIELD OF INVENTION

The invention relates to a frangible fixing screw. Such a screw is generally designed for the biomedical industry. It is particularly adapted for attaching a superstructure to an intra-osseous implant, particularly a periodontal implant. It is more specifically but not exclusively adapted to the installation of superstructures on such implants when they are made of a ceramic material, particularly zirconia. In one particularly advantageous application, this invention makes it possible to use a screw that is itself made of a ceramic material, particularly yttria-stabilized zirconia. The screw of the invention can also be used as a permanent or temporary intra-osseous implant, in particular for the fixing of osteosynthesis platesintra-osseous.

### BACKGROUND OF THE INVENTION

The patent EP 1 034 750-B1 describes a device for attaching a superstructure on an intra-osseous implant comprising an inner thread. Such a device, called a transfixing device, is used commonly, particularly for attaching abutments, or piers, on a periodontal implant, which abutments act as supports for crowns. According to this prior art, attaching was carried out by a transfixing screw including a screw head comprising a bearing surface and a thread, wherein the head of the screw is connected to the thread by a groove. The end of the thread near the screw head comprises two flat sections. Thus, in the event of a violent impact on the crown, the groove of the screw constitutes a frangible zone and breaks to limit the force transmitted to the implant, so as to avoid damaging the bone on which it is implanted. The flat sections make it possible to remove the said screw after the head of the screw is separated from the thread following such an event.

Protecting the implant and the bone tissue in the event of a large force or impact on the superstructures is particularly important when said implant is made of a ceramic material, with high elastic modulus and hardness. That is because in such conditions, the difference between the elastic modulus of the bone and that of the implant leads to elastic deformation incompatibility stresses between the bone and the implant, which remain limited in usual living conditions but can become great in extreme situations and lead to a break in the tissue and/or the implant. The hardness of the implant renders its removal problematic once it has been integrated into the bone, making it indispensable to retain the integrity of the implant in all circumstances, including these extreme circumstances. As the price of such a safety, making a groove that is too deep would create an excessively weakened frangible section and a zone that would be particularly vulnerable to fatigue stress. Such fatigue stress may occur, for example, in periodontal implant systems, during mastication or when the subject who wears the implant grinds its teeth. Now, the creation of a groove as described in the prior art does not merely lead to the local reduction of the resistant section, which is desirable for making a frangible zone, but also a stress concentration coefficient Kt which affects all the stress modes of that part and produces an adverse effect, including for stress where the level is significantly lower than that leading to a break. Thus, in this example of the prior art, the stress concentration coefficient Kt relating to the presence of the groove is located between 1.60 and 2.1 depending on the mode of stress loading, meaning that it is of the same order of magnitude as what differentiates usual stresses and strains, to which it must resist, particularly in fatigue, from exceptional loads under which it must break. The problem is even more critical when the screw is made in material such as a ceramic material that does not have a significant capacity of accommodation by plastic deformation, so as to slow or to stop the progression of cracks, and where the fatigue resistance ratio in relation to static resistance is low.

### OBJECT AND SUMMARY OF THE INVENTION

In order to solve these prior art drawbacks, the invention discloses a screw, particularly for attaching a superstructure to an intra-osseous implant, said screw extending along a longitudinal axis and comprising:
a. a head comprising a bearing surface able to cooperate with a bearing surface of the superstructure to hold said superstructure in position;
b. a thread able to engage with a tapping so that the bearing surfaces of the superstructure and of the screw can be clamped together;
c. said screw includes, between the bearing surface and the thread, a portion, called the safety portion, comprising a functional shape, called the driving shape, and a frangible section at the connection between said safety portion and the head of the screw, so that the driving shape remains attached to the thread if the frangible section breaks.

Thus, the screw in question includes a special breaking or frangible zone under the head of the screw, which makes it possible to keep the functional shape, called the driving shape, intact if the frangible zone breaks. In the absence of a groove, the stress concentration coefficient is clearly reduced and enables the section to play its part as a frangible zone, without overly affecting the fatigue strength of said zone with regard to usual strains and stresses. The functional shape is called the driving shape because in most applications, this shape makes it possible to drive the screw to loosen it. However, said shape may also be used for other purposes, particularly for centering, supporting or sealing a superstructure without departing from the scope of this invention.

The invention can be implemented in the advantageous embodiments described below, which may be considered individually or in any technically operative combination.

According to a first embodiment of the screw in the invention, the safety portion is conical in relation to the longitudinal axis, wherein the smaller section of said portion is located near the bearing surface of the head and the larger section of said portion is smaller than or equal to the section at the root of the thread, wherein the smaller section is the frangible section. Thus the section of the driving shape near the thread is equal to or larger than the section at the root of the thread, and so it eliminates the risk of breaking the driving shape at its connection with the thread and thus ensures that said driving shape is retained after a possible break in the frangible section.

According to a second embodiment of the screw in the invention, the safety portion comprises an internal hollow that is capable of creating a frangible zone at the connection between the safety portion and the head. This embodiment simplifies the shape of the screw and makes it possible to retain a driving shape with a constant perimeter over its entire height when this driving shape is prominent in relation to the implantation of the thread.

The two embodiments may be combined.

Advantageously, the screw comprises a connection with a gradual section between the small section of the driving shape and the head of the screw. That gradual connection makes it possible to reduce the stress concentration coefficient between the frangible section and the head of the screw.

Advantageously, the driving shape is prominent in relation to the implantation of the thread. This embodiment is particularly advantageous when the screw is used as a transfixing element, as the thread of said screw is located in the tapping of an intra-osseous implant. Thus, the driving shape remains easily accessible for removing the screw in the event of a break, when the implant is implanted in vivo.

According to an advantageous embodiment, the driving shape is a polygonal shape. It can thus be easily driven using a key. Unlike the prior art represented by the European patent EP 1 034 750-B1, where the flat sections on the threaded part only allow two angular positions of the key, when loosening is initiated in the event of a break, the polygonal shape allows at least four positions of the driving key or more, depending on the number of sides of the polygon, which is advantageous when the implant is in a location that is difficult to access, for example in the case of a periodontal implant.

Advantageously, the conical angle of the driving shape ranges between 5° and 6°. That conical angle value provides a particularly advantageous compromise between the length of the driving shape and the effect of the reduction of the section.

According to an advantageous embodiment, the ratio of the surfaces of the small section and the large section of the conical part ranges between 0.75 and 0.9. That range of ratios provides the best compromise between the static resistance and the fatigue resistance regardless of the material, particularly metal or ceramic, of which the screw is made.

According to an alternative embodiment, the driving shape of the safety portion comprises conical faces from a base that is polygonal in section and lugs that protrude from said faces, wherein said lugs extend without tapering along the axial direction of the screw. This alternative makes it possible to retain a driving shape with a constant perimeter over the entire height of the safety portion, while allowing a gradual reduction in the section of said safety portion.

According to a particular embodiment, adapted to the case where the screw comprises a hollow in the frangible section, said hollow constitutes a driving shape. Thus, the screw can be shorter, with equivalent functionality.

According to an advantageous alternative of this particular embodiment of the screw in the invention, the hollow is an inner tapping. This alternative embodiment is particularly suited to the cases where the screw itself is implanted in the conjunctive tissue. Thus, it is possible, if the head of the screw is broken accidentally or deliberately, first, to precisely locate the breaking zone in the frangible zone, and secondly, to use the hollow that is thus revealed to reinstall a fastening element or as a sealing base.

Advantageously, the screw of the invention is made in stainless steel. That material offers maximum safety with regard to fatigue stresses.

Alternatively, the screw of the invention is made in yttria-stabilized zirconia. The addition of yttria increases the fatigue resistance of zirconia, and the particular design of the screw makes that material appropriate for such use, and thus creates an intra-osseousimplantation device associated with superstructures where the whole is free of metal.

The invention also relates to a method for manufacturing a screw according to the embodiments of the invention comprising a hollow, which method includes a step consisting in obtaining a blank of said screw by an additive machining process. This type of method is used advantageously by building up the volume in successive layers to make any form of hollow.

The invention also relates to a key comprising a conical recess that is complementary with the driving shape of a screw according to any of the preceding claims and able to drive said screw to rotate around the longitudinal axis when the connection between the head of the screw and the driving shape is broken.

### BRIEF DESCRIPTION OF THE DRAWINGS

The invention is described below in its preferred embodiments, which are not limitative in any way, and by reference to FIGS. 1 to 4, wherein:

FIG. 1 represents a front view of an exemplary embodiment of a transfixing screw according to the invention;

FIG. 2 is a front view along a section C-C, defined in FIG. 1, of an exemplary embodiment of a screw according to the invention comprising an internal hollow at the connection between the safety portion and the head of the screw;

FIG. 3 is a perspective view along a section B-B defined in that same view of an exemplary embodiment of a screw according to the invention, comprising a safety portion with a driving shape with lugs; and

FIG. 4 is a view of a longitudinal section (C-C) of a screw according to an embodiment of the invention where the internal hollow also carries out the function of a driving shape, in FIG. 4A in the form of a conical tapping, and in FIG. 4B, in the form of a polygonal recess in a partial view Z defined in FIG. 4A.

### DETAILED DESCRIPTION OF THE EMBODIMENTS

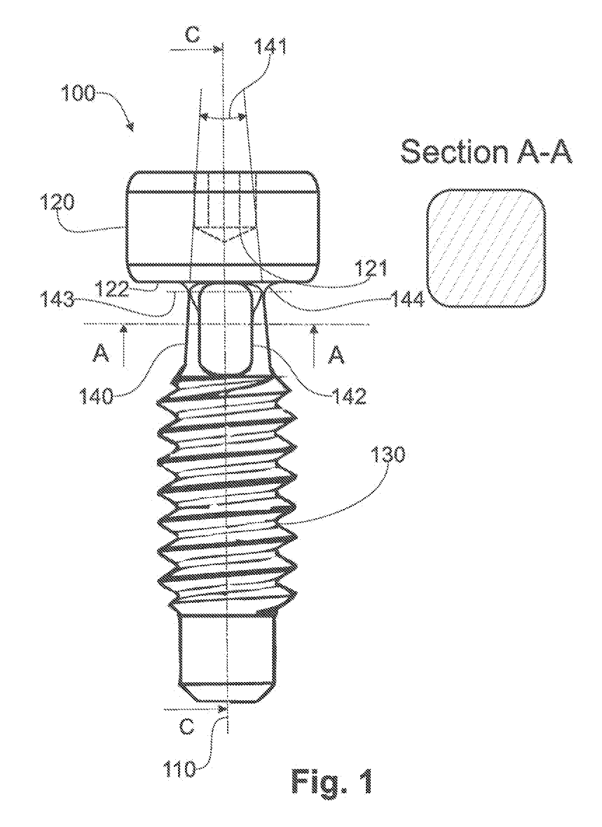

In FIG. 1 according to a first exemplary embodiment, the fixing screw (100) of the invention comprises a screw head (120) and a threaded part (130) that are coaxial along a longitudinal axis (110). The screw head (120) comprises a driving shape (121), for example in the shape of a hexagonal recess. The other end of the head (120), near the thread (130), comprises a bearing surface (122), which, in this exemplary embodiment, is a flat surface but which may be conical, spherical or have any other shape capable of creating a contact surface in order to allow tightening on an adapted complementary surface on a superstructure. The threaded part (130) of the screw is inserted in the tapping of an intra-osseous implant, after said implant has been implanted in the receiving tissue. The bearing surface (122) of the screw head (120) and the thread (130) thus cooperate to hold a superstructure such as an abutment in place on said implant by tightening.

Between the bearing surface (122) and the start of the thread (130), a safety portion (140) comprises a driving shape (142), for example a polygonal shape. In this first exemplary embodiment of the screw in the invention, this safety portion (140) is conical in shape, where the small section is close to the screw head, and where the polygonal driving shape follows the conical shape of said safety portion. In one exemplary embodiment, section AA, the polygon is a square.

The conical angle (141) of this safety zone advantageously ranges between 5° and 6°. As a result of that conical angle, there is a small section (143) between the thread and the head of the screw, wherein the surface of that section is between 0.75 and 0.9 times the surface of the connection section between the safety portion (140) and the thread.

Advantageously, the end of the safety portion comprises a gradual connection, for example in the form of a connecting radius (144). This characteristic makes it possible to limit the stress concentration at the connection with the bearing surface (122) and thus provides the presence of a zone (143) that is frangible in the event of exceptional stresses, without affecting the fatigue resistance of the part.

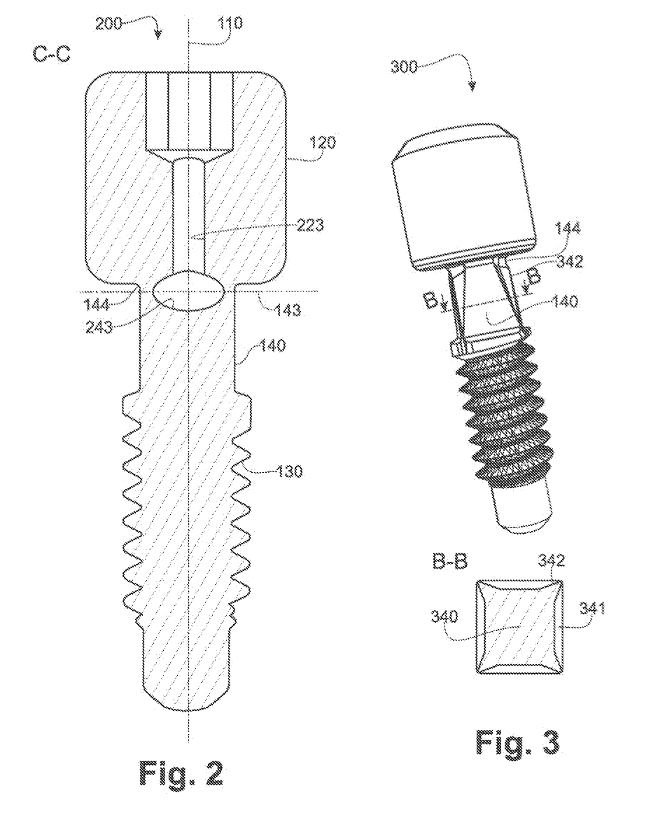

In FIG. 2 of a second embodiment of the screw (200) according to the invention, the frangible zone (143) is obtained by making a hollow (243) inside the safety portion (140), wherein said safety portion is not conical in this exemplary embodiment. That hollow is made by machining, or when the screw (200) is molded. In that last mode of manufacturing, the hollow is obtained by inserting a core, or insert, in the injection mold. In an embodiment adapted to screws made of a sintered ceramic material, said core is of the calcinating type and disappears when the screw is sintered. If the hollow (243) is achieved by machining, such machining is carried out when the ceramic material is green, before sintering, if the screw is made of such a material. Advantageously, such machining uses a so-called recessing tool, that is to say a rotating tool with a body bearing machining grains extending radially, where the eccentricity of said grains in relation to the body can be modified during the machining process. Such a tool makes it possible to drill a first bore (223) with a diameter clearly smaller than that of the hollow (243) and then to make the said hollow by moving the boring grains off center.

In an alternative embodiment, such a hollow (243) may be combined with a conical safety portion (140) to make the frangible section (143).

The hollow is revealed when the frangible zone (143) breaks. Thus, said hollow may also be used for functional purposes after the frangible zone has broken. As a non-limitative example, the hollow (243) and the prominent safety portion (140) cooperate to install a superstructure, where said superstructure is centered on the prominent portion and sealed on it, for example with cement or resin poured into the hollow (243). In this example, the safety portion (140) is advantageously conical to facilitate the centering of the superstructure on it during installation after the frangible zone has broken.

In FIG. 3, the section of the safety portion (140) is not limited to a polygonal shape and may have any section adapted to a driving shape function. As non-limitative examples, the section of the safety portion (140) may have a multilobed shape or be a curvilinear polygon. In one exemplary embodiment of the screw (300) of the invention, the driving shape (340) of the safety portion (140) comprises conical faces (341) with a base that is polygonal in section and lugs (342) that protrude from said faces (341), wherein said lugs extend without tapering along the axial direction (110). The key used to drive the shape moves the protruding lugs, so that it is not necessary for the driving recess of said key to be conical.

According to an alternative embodiment, the screw (100, 200, 300) is made of stainless steel, for example AISI 316L, in a super strain hardened state.

This is an austenitic stainless steel, typically comprising 18% chromium and 10% nickel, with a carbon content below 0.03%. Because of the low carbon content, the steel cannot be hardened using thermal treatment, and strain hardening makes it possible to considerably improve its mechanical characteristics. Alternatively, a ferritic stainless steel of the F16PH type may also be used for its resistance to corrosion and its mechanical characteristics.

In a preferred embodiment, said screw (100, 200, 300) is constituted of a ceramic material, particularly yttria-stabilized zirconia. (ZrO₂, 3Y₂O₃). Even though it does not match the fatigue resistance characteristics of a metallic materials, the presence of yttrium oxide slows down the propagation of cracks. The combination of this characteristic, intrinsic to the material, with the structural characteristics of the screw of the invention, makes it possible to make an assembly with an implant, a superstructure and a transfixing screw in a ceramic material, particularly zirconia, very safely with respect to the risk of an implant breakage, protected by the frangible zone of the screws, and to take full advantage of the advantageous biocompatibility, longevity and colour of the material for such applications.

According to exemplary embodiments in FIGS. 1 to 3, the safety portion (140) is prominent in relation to the implant when the screw (100, 200, 300) is screwed into the internal tapping of said implant. Thus, if the frangible section (143) breaks, the superstructure is released and the driving shape protrudes out. It is then easy to extract said screw from the implant by simply unscrewing it with a key of the box spanner type, with a recess having a hollow shape that is complementary to that of the safety portion (140).

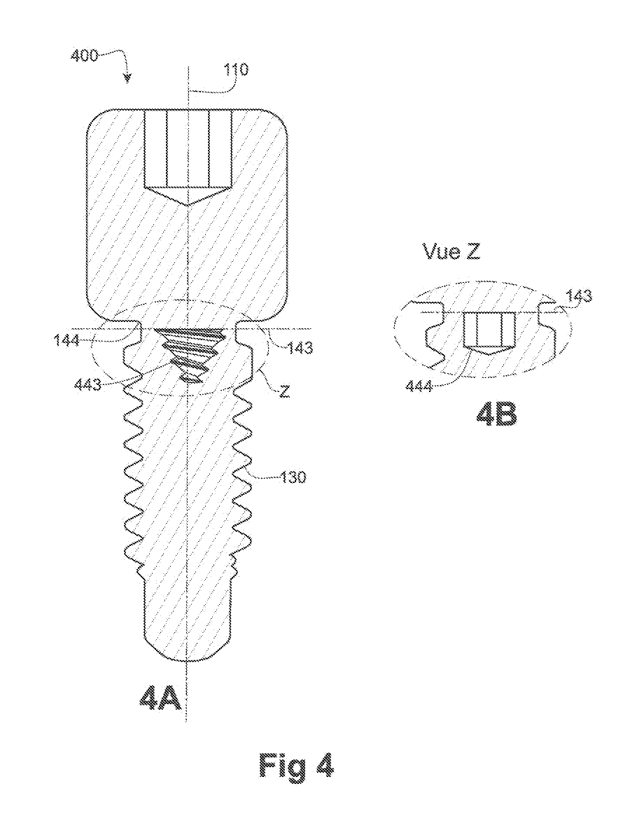

In FIG. 4, according to another exemplary embodiment, the safety portion has a reduced height, and the frangible zone (143) is placed close to the start of the thread (130). In this exemplary embodiment, the hollow (443, 444) has the simultaneous functions of creating the frangible zone and constituting a driving shape that remains connected to the thread after said frangible zone (143) has broken. In one exemplary embodiment in FIG. 4B, the hollow (444) has a polygonal shape. This embodiment makes it possible to reduce the height of the screw (400), particularly for transfixing applications. In another exemplary embodiment in FIG. 4A, the hollow (443) is a tapping, for example a conical tapping. That last embodiment is particularly suitable for a screw (400) implanted directly into conjunctive tissue, particularly for attaching a prosthesis or an osteosynthesis plate. According to this embodiment, the presence of the frangible zone may be used advantageously to deliberately break the screw head. The hollow, which is revealed upon the break, makes it possible to attach the superstructures on the portion of the screw that is bio-integrated into the tissue.

According to these embodiments of the screw (400) of the invention shown in FIG. 4, the screw is advantageously manufactured using an additive machining method. Such a method uses a screw (400) construction in successive layers and thus makes it possible to make any form of hollow that does not go through. Methods such as laser sintering or laser projection fusion are particularly suitable for this embodiment, whether the screw is made of metal or of a ceramic material.

The description and the exemplary embodiment above clearly show that the invention achieves its objectives, particularly the screw according to the invention known as a frangible screw makes it possible to define a frangible zone (143) that can break under a predefined stress while retaining the functions of the broken part that is not released by the break. These functions can particularly make it easy to remove that unreleased part, or to use that portion for fixing or sealing.

1. A screw for attaching a superstructure to an intra-osseous implant, said screw extending along a longitudinal axis and comprising:
a head comprising a bearing surface configured to cooperate with a bearing surface of the superstructure to hold the superstructure in position;
a thread configured to engage with a tapping of the implant to tighten the bearing surfaces of the superstructure and the screw; and
wherein the screw comprises, between the bearing surface and the thread, a safety portion comprising a driving shape and a frangible section at the connection between the safety portion and the head of the screw, so that the driving shape remains attached to the thread when the frangible section breaks.

2. The screw according to claim 1, wherein the safety portion is conical in relation to the longitudinal axis comprises a small section and a large section, and wherein the small section of the safety portion is located near the bearing surface of the head and the large section of the safety portion is smaller than or equal to a root section of the thread, wherein the small section being the frangible section.

3. The screw according to claim 1, wherein the safety portion comprises an internal hollow configurable to provide the frangible section at the connection between the safety portion and the head.

4. The screw according to claim 2, further comprising a connection with a gradual section of the safety portion and the bearing surface of the head.

5. The screw according to claim 1, wherein the driving shape is prominent in relation to an implantation of the thread.

6. The screw according to claim 1, wherein the driving shape is a polygonal shape.

7. The screw according to claim 2, wherein a conical angle of the driving shape ranges between 5° and 6°.

8. The screw according to claim 2, wherein the ratio between the area of the small section and the large section of the safety portion ranges between 0.75 and 0.9.

9. The screw according to claim 2, wherein the driving shape of the safety portion comprises conical faces from a base that is polygonal in section and lugs that protrude from said faces, said lugs extending without tapering along an axial direction of the screw.

10. The screw according to claim 3, wherein the hollow constitutes a driving shape.

11. The screw according to claim 10, wherein the hollow is an inner tapping.

12. The screw according to claim 1 is made of stainless steel.

13. The screw according to claim 1 is made of yttria-stabilized zirconia.

14. A method for manufacturing a screw according to claim 3, comprising the step of obtaining a blank of the screw comprising the hollow by an additive machining process.

15. A key comprising a shape that is complementary with the driving shape of a screw according to claim 1 and configured to drive said screw to rotate around the longitudinal axis when the connection between the head of the screw and the safety portion is broken.
